(19) United States
(12) Patent Application Publication
(10) Pub. No.: US 2016/0287529 A1
(43) Pub. Date: Oct. 6, 2016

(54) METHOD FOR TREATING AND PREVENTING RECURRENT INTERDIGITAL TINEA PEDIS (ATHLETE'S FOOT) DISEASE

(71) Applicant: Kenneth W. Wright, Los Angeles, CA (US)

(72) Inventor: Kenneth W. Wright, Los Angeles, CA (US)

(21) Appl. No.: 14/673,179

(22) Filed: Mar. 30, 2015

(51) Int. Cl. A61K 31/045; A61K 9/00

(52) U.S. Cl. CPC A61K 31/045; A61K 9/0014

(57) ABSTRACT

A solution and a method by which the solution is used to treat and prevent recurrent interdigital tinea pedis (athlete's foot). The solution contains a high concentration alcohol which serves as an antifungal agent and a fragrance dissolved in the alcohol which serves as a deodorant and masks the alcohol scent in public. The solution is topically applied (e.g., sprayed) on a user's wet foot and between his toes. The alcohol in the solution evaporates to remove the moisture between the toes so as to combat the athlete's foot disease. In a preferred embodiment, the solution has a concentration of alcohol lying in a range of between 70% to 90% and a concentration of fragrance lying in a range between 0.5% to 1.0%. The remainder of the solution is ideally water.

### BACKGROUND OF THE INVENTION

1. Field of the Invention

This invention relates to a low cost, topical solution and to a method for using the solution to treat and prevent recurrent athlete's foot disease. The solution, which contains a mixture of high concentration alcohol and fragrance, is applied (e.g., sprayed) from a dispenser to the wet foot and between the toes of a user to dry the user's moist skin and thereby combat the athlete's foot fungal infection.

2. Background Art

Interdigital tinea pedis (commonly known as “athlete's foot”) is a fungal infection of the skin that is associated with wet, macerated interdigital spaces, with fissuring of the interdigital space and hyperkeratosis, leukokeratosis and erosions. Athlete's foot is typically transmitted in moist environments, such as showers, bathhouses, and locker rooms. In particular, athlete's foot is caused by moisture between the toes which promotes growth of fungi in the genus Trichophyton, including Trichophyton rubrum, T. mentagrophytes, and Epidermophyton floccosum. Bacteria can also cause a secondary super infection of the skin contributing to the athlete's foot disease. The fungus grows best in moist skin areas such as between the toes. Athlete's foot has heretofore been treated by a number of pharmaceutical and anti-fungal applications. Despite these conventional treatments, the athlete's foot fungal infection will frequently return.

The best practice guidelines of the American Academy of Dermatology recognizes that moisture between one's toes contributes to athlete's foot. A significant problem in avoiding contracting athlete's foot is the difficulty in drying moisture between the toes after sweating, showering, bathing and swimming. The inability to dry between the toes predisposes the individual to athlete's foot, and susceptibility to the disease is exacerbated when closed toed shoes are worn in which case moisture on the foot and between toes is trapped.

Accordingly, there exists a need for a low cost, practical and efficacious preventive treatment of athlete's foot disease that can be prescribed over the counter and used on a chronic basis. The treatment of athlete's foot disease has typically been based on the use of a topical antifungal spray, powder, cream or gel. However, these treatments do not specifically address prevention. Fundamental to preventing athlete's foot disease is being able to eliminate excess moisture between the toes which is retained after sweating, showering, bathing and swimming. Foot powders attempt to dry the toes, however, they are marginally effective and have the drawback of leaving excess powder deposits on the user's skin, clothing, shoes and surroundings. Moreover, the use of foot powder has not proven entirely effective against chronic recurrent athlete's foot disease.

Common antifungal medications in over-the-counter athlete foot remedies including miconazole nitrate and tolnaftate are effective for treating athlete's foot but are not always effective for preventing either new or the recurrence of the disease. Consequently, these medications have had only limited success in combating athlete's foot disease because, if used chronically, resistant organisms will still occur. The Cochrane Database Syst Rev., 2007, reported the pooled relative risks of failure to cure (RRFC) for topical treatments for fungal infections of the skin and nails of the human foot. The Cochrane study found that even the best anti-fungal topical drugs (i.e., the allylamines) had on average a 30% failure to cure. Other anti-fungal topical drugs (e.g., azoles, undecenoic acid, and tolnaftate) were less effective. Even when topical anti-fungal treatment is effective, the recurrence rate has been found to be high. Presently, recurrent, or what podiatrists sometimes call “chronic recurrent,” athlete's foot remains a significant unresolved disorder.

### SUMMARY OF THE INVENTION

In general terms, a low cost solution and a method are disclosed herein for treating and preventing recurrent interdigital-type tinea pedis (i.e., athlete's foot). The method includes the steps of creating a solution by mixing water with a high concentration alcohol (e.g., isopropyl or ethanol) and a liquid fragrance (e.g., baby powder or fruit) which is dissolved in the alcohol. The solution is then topically applied (e.g., sprayed) from a dispenser to the foot and between the toes of a user to dry the user's moist skin and thereby combat the athlete's foot fungal infection.

According to a preferred embodiment, the solution contains the fragrance in a concentration range of approximately 0.5% to 1.0% dissolved in the alcohol in a concentration range of approximately 70% to 90%. The alcohol provides the solution with an anti-fungal agent to disinfect between the user's toes. The fragrance masks the alcohol scent and acts as a deodorant to promote public acceptance. The alcohol causes any water between the user's toes to be dissolved and evaporated so as to leave the skin between the toes dry and thereby avoid fungal infection.

### BRIEF DESCRIPTION OF THE DRAWINGS

There are no drawings for this application.

### DESCRIPTION OF THE PREFERRED EMBODIMENT

In accordance with a preferred embodiment, a low cost solution and a method are described below for treating and preventing recurrent interdigital-type tinea pedis (i.e., athlete's foot) disease by which the solution, containing a mixture of high concentration alcohol and a suitable dose of fragrance, is topically applied to the foot and toes of a user. This solution has a significant antimicrobial characteristic and the ability to dry moist skin of the foot and between the toes so as to advantageously prevent the athlete's foot disease. The addition of the fragrance to the high concentration alcohol component masks the alcohol scent and mitigates awareness of the alcohol-based solution in public. In this same regard, the fragrance also advantageously acts as a foot deodorant.

The solution includes a fragrance in a concentration range of approximately 0.5% to 1.0% dissolved in a high concentration of alcohol such as isopropyl or ethanol in a preferred range of approximately 70% to 90%. The remaining component of the solution is ideally water. The fragrance is preferably alcohol soluble such as, for example, baby powder or fruit that is mixed with the alcohol to form a solution that is capable of being sprayed from a dispenser. By way of particular example, the fruit fragrance is derived from a liquid fruit extract diluted by water. The baby powder fragrance is derived from a water-soluble baby powder scented oil. The mixture containing water, alcohol and the fragrance is a clear volatile solution that will not stain skin, the user's clothes, or the surrounding environment when applied topically to the foot. Unlike powder which often leaves a messy environment, the alcohol solution evaporates and disappears quickly without leaving behind a residue of particulate matter on surfaces. That is, the method disclosed herein relies on the characteristic of alcohol as a solvent that dissolves water. The alcohol and fragrance solution can be topically applied by being sprayed on the wet skin of the foot and specifically at hard-to-access areas between the toes. Because the alcohol solution dissolves water and then evaporates, the solution removes moisture from between the toes. In other words, the alcohol evaporates to leave the skin between the toes dry and thereby prevents the athlete's foot disease.

In addition, the antiseptic characteristic of high concentration alcohol of the solution acts as an antifungal and antibacterial agent to resist infection and prevent the return of the athlete's foot disease. High concentration alcohol, such as ethanol and isopropyl alcohol, when used topically is well tolerated by the skin. Thus, it may be appreciated that the application of the high alcohol content solution herein described prevents athlete's foot disease by a combination of contact antimicrobial action and drying the skin of the user's foot and between his toes. The fragrance in the alcohol solution reduces the alcohol scent so as to improve public acceptance of the solution and offer a foot deodorant function.

Dilution trials conducted by the applicant herein, Kenneth W. Wright, MD, determined the concentration of fragrance to be optimal at the above-identified range of 0.5% to 1% which is sufficient to mask the alcohol smell when the user is out in public. The clinical trials were conducted on 10 consecutive adult patients with a history of recurrent athlete's foot disease. The results of these trials showed a significant reduction and recurrence of disease in all patients who applied a daily dose of the high concentration alcohol solution described above. The alcohol/fragrance solution herein disclosed was found to be effective for preventing athlete's foot skin disease by drying moisture on the user's foot and between his toes, disinfecting between the toes, and deodorizing the user's foot by masking the alcohol smell.

1. A solution to be applied to a user's foot and between his toes to treat and prevent recurrent athlete's foot disease, said solution including primarily alcohol and a fragrance dissolved in the alcohol to mask the scent thereof.

2. The solution recited in claim 1, wherein said solution has a concentration of alcohol lying in a range of between 70% to 90%.

3. The solution recited in claim 1, wherein said solution has a concentration of fragrance lying in a range between 0.5% to 1.0%.

4. The solution recited in claim 1, wherein said solution has a concentration of alcohol lying in a range of between 70% to 90% and a concentration of fragrance lying in a range between 0.5% to 1.0%.

5. The solution recited in claim 1, wherein said alcohol is isopropyl alcohol.

6. The solution recited in claim 1, wherein said alcohol is ethanol alcohol.

7. The solution recited in claim 1, wherein said fragrance is alcohol soluble.

8. The solution recited in claim 1, wherein said fragrance is selected from a group of fragrances including a liquid fruit extract diluted by water and a water-soluble body powder scented oil.

9. A method for treating and preventing recurrent athlete's foot disease comprising the step of topically applying a solution to the foot and between the toes of a user, wherein said solution includes primarily alcohol and a fragrance dissolved in the alcohol to mask the scent thereof.

10. The method recited in claim 9, wherein said solution includes a concentration of alcohol lying in a range of between 70% to 90% and a concentration of alcohol-soluble fragrance lying in a range of between 0.5% to 1.0%.

11. The method recited in claim 9, comprising the additional step of spraying the solution on the foot and between the toes of the user.
